(19) United States
(12) Patent Application Publication
(10) Pub. No.: US 2025/0345481 A1
(43) Pub. Date: Nov. 13, 2025

(54) NANOCOMPOSITE HYDROGELS AND RELATED METHODS AND APPLICATIONS

(71) Applicant: The Chinese University of Hong Kong, Shatin (HK)

(72) Inventors: Ling Qin, Lu Tzs Tin (HK); Jiaxin Guo, Shatin (HK); Jiankun Xu, Shatin (HK)

(73) Assignee: The Chinese University of Hong Kong, Shatin (HK)

(21) Appl. No.: 18/658,479

(22) Filed: May 8, 2024

(51) Int. Cl. A61L 27/26; A61K 9/00; A61K 9/06; A61K 47/02; A61K 47/34; A61K 47/42; A61L 27/02; A61L 27/22; A61L 27/52

(52) U.S. Cl. CPC A61L 27/26; A61K 9/0024; A61K 9/06; A61K 47/02; A61K 47/34; A61K 47/42; A61L 27/025; A61L 27/227; A61L 27/52; A61L 2400/06; A61L 2400/12; A61L 2430/02

(57) ABSTRACT

Nanocomposite hydrogels suitable for bone tissue regeneration may include (i) a scaffold comprising serum albumin and a cell adhesion promoter crosslinked with polyethylene glycol and (ii) a nanoparticle dispersed in the scaffold. Said nanocomposite hydrogels may formed from an injectable composition that includes: a nanocomposite hydrogel precursor A comprising that comprises a polyethylene glycol with two or more N-hydroxysuccinimide-terminal groups (PEG-NHS); and a nanocomposite hydrogel precursor B comprising a serum albumin, a nanoparticle, and a cell adhesion promoter; wherein the nanocomposite hydrogel precursor A and the nanocomposite hydrogel precursor B are physically separated.

### FIELD

The present disclosure relates hydrogels, specifically nanocomposite hydrogels, suitable for bone tissue regeneration including related compositions and methods.

### BACKGROUND

Hydrogels are polymer scaffolds that can mimic the extracellular matrix (ECM) and be used to carry various cells and bioactive factors to fill defects and promote tissue regeneration. Tissue engineering applications use hydrogel scaffolds to seed cells or composite bioactive growth factors to promote tissue repair and regeneration. For example, some studies have shown that hydrogels encapsulating stem cells have a better effect on promoting bone tissue repair. The hydrogel gradually dissolves as surrounding tissue grows in, thereby avoiding the complexity of surgical removal and reducing chronic inflammatory reactions.

Hydrogels with adjustable crosslinking time (or gelation time) may be advantageous for various situations so that the hydrogels can be employed as materials for injecting and filling different tissues for regeneration. However, the adjustment of traditional gelation time is usually limited to changing the concentration of gel precursor and/or crosslinker, which can be difficult to accurately quantify. Improper gelation rate can lead to over-implantation, drug leakage, and increased side effects. Accordingly, there is a need for hydrogels with consistent gelation time that can be readily varied and tailored to the desired applications.

### BRIEF SUMMARY

A nonlimiting example injectable composition may comprise: a nanocomposite hydrogel precursor A comprising that comprises a polyethylene glycol with two or more N-hydroxysuccinimide-terminal groups (PEG-NHS); and a nanocomposite hydrogel precursor B comprising a serum albumin, a nanoparticle, and a cell adhesion promoter; wherein the nanocomposite hydrogel precursor A and the nanocomposite hydrogel precursor B are physically separated; and wherein the nanocomposite hydrogel precursor A and the nanocomposite hydrogel precursor B are capable of forming a nanocomposite hydrogel when mixed.

A nonlimiting example method may comprise: injecting a nanocomposite hydrogel precursor A and a nanocomposite hydrogel precursor B into a space in a biological environment, thereby causing mixing thereof and formation of a nanocomposite hydrogel in the space, wherein the nanocomposite hydrogel precursor A comprises a polyethylene glycol with two or more N-hydroxysuccinimide-terminal groups (PEG-NHS), and wherein the nanocomposite hydrogel precursor B comprises a serum albumin, a nanoparticle, and a cell adhesion promoter.

Another nonlimiting example method may comprise: mixing a nanocomposite hydrogel precursor A and a nanocomposite hydrogel precursor B to form a nanocomposite hydrogel, wherein the nanocomposite hydrogel precursor A comprises N-a polyethylene glycol with two or more N-hydroxysuccinimide-terminal groups (PEG-NHS), and wherein the nanocomposite hydrogel precursor B comprises a serum albumin, a nanoparticle, and a cell adhesion promoter.

A nonlimiting example hydrogel composition may comprise: a scaffold comprising serum albumin and a cell adhesion promoter crosslinked with polyethylene glycol; and a nanoparticle dispersed in the scaffold.

### BRIEF DESCRIPTION OF THE DRAWINGS

FIG. 1 illustrates a proposed mechanism for forming nanocomposite hydrogels according to at least some embodiments of the present disclosure.

FIG. 2 illustrates photographs and scanning electron micrographs corresponding to control hydrogels and nanocomposite hydrogels according to at least some embodiments of the present disclosure.

FIG. 3A illustrates the method for ascertaining the gelation time.

FIG. 3B illustrates a plot of the gelation time for nanocomposite hydrogels according to at least some embodiments of the present disclosure as a function of the magnesium oxide nanoparticle concentration where the inlay plot is the 15-30 mg/mL data enlarged.

FIG. 3C illustrates photographs during the gelation time method for control hydrogels and nanocomposite hydrogels according to at least some embodiments of the present disclosure.

FIG. 4A illustrates a fluorescent image after treating with live/dead imaging for cells after the 3-day incubation period with control hydrogels and nanocomposite hydrogels according to at least some embodiments of the present disclosure.

FIG. 4B is a bar graph of the number of adherent cells after the 3-day incubation period with control hydrogels and nanocomposite hydrogels according to at least some embodiments of the present disclosure.

FIG. 4C is a bar graph of the percentage of total cells that are living after the 3-day incubation period with control hydrogels and nanocomposite hydrogels according to at least some embodiments of the present disclosure.

FIG. 5A is X-ray images of the whole bone, FIG. 5B is three-dimensional micro-CT image of the drilled hole, taken starting from the 4th week after either no implantation, implantation of a control hydrogel, or implantation of a nanocomposite hydrogel according to at least some embodiments of the present disclosure into a bone defect.

FIG. 6 illustrates three-dimensional reconstructions of angiography based on micro-CT.

FIGS. 7A-7C are bar graphs of the statistical analysis of the micro-CTs of FIG. 6. Specifically, FIG. 7A is a bar graph of the percentage of blood vessels based on total volume of the defect area. FIG. 7B is a bar graph of the number of blood vessels based the defect area. FIG. 7C is a bar graph of average thickness of the blood vessels.

### DETAILED DESCRIPTION

The present disclosure relates hydrogels, specifically nanocomposite hydrogels, suitable for bone tissue regeneration. The nanocomposite hydrogels of the present disclosure use nanoparticles (e.g., bioactive nanoparticles) to trigger the crosslinking reaction, thereby controllably adjusting the gelation time of the nanocomposite hydrogel. Advantageously, the nanoparticles may dissolve over time where, when using a bioactive nanoparticle, the dissolution products promote biological processes such as osteogenesis, angiogenesis, antibacterial, and inhibition of inflammation in the body, aiming to achieve better tissue regeneration effects. Therefore, the nanocomposite hydrogels of the present disclosure may be widely used in the field of tissue regeneration and repair including for implant and filling microsurgeries.

The nanocomposite hydrogels of the present disclosure are formed by mixing a nanocomposite hydrogel precursor A with a nanocomposite hydrogel precursor B. The nanocomposite hydrogel precursor A includes an N-hydroxysuccinimide-terminal polyethylene glycol (PEG-NHS) that crosslinks a serum albumin present in the nanocomposite hydrogel precursor B after mixing the two precursors.

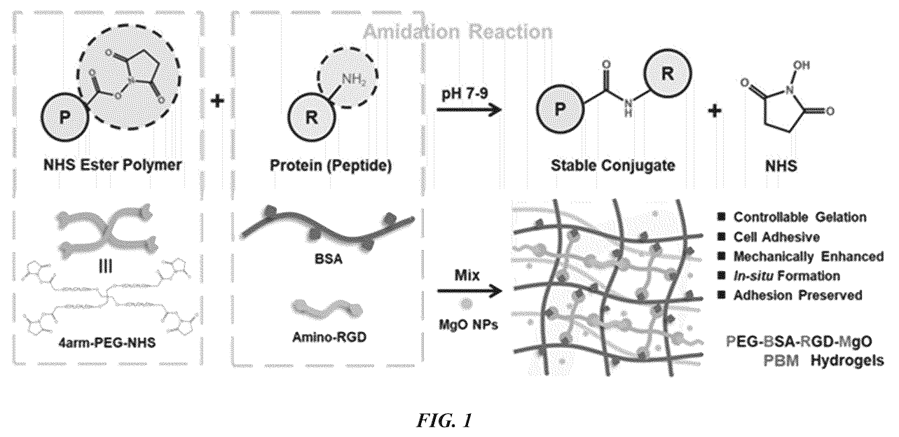

The nanocomposite hydrogel precursor A may comprise an N-hydroxysuccinimide-terminal polyethylene glycol in a carrier fluid. The nanocomposite hydrogel precursor B may comprise a serum albumin, a cell adhesion promoter, and a nanoparticle in a carrier fluid. FIG. 1 illustrates a nonlimiting proposed mechanism for forming nanocomposite hydrogels according to at least some embodiments of the present disclosure. Without being limited by theory, the PEG-NHS may crosslink serum albumin and cell adhesion promoter to form the hydrogel scaffold.

Further, without limitation by theory, crosslinking (or gelation) is believed to be a pH-sensitive reaction, which can be facilitated by the nanoparticles in the nanocomposite hydrogel precursor B. Preferably, the concentration of the nanoparticles in the nanocomposite hydrogel precursor B can be used to tailor the pH of the nanocomposite hydrogel precursor B and the gelation time of the nanocomposite hydrogels. The cell adhesion promoter is believed to improve the cell bioadhesive and osteogenic properties of nanocomposite hydrogels.

The resultant nanocomposite hydrogel comprises (i) a scaffold comprising serum albumin and cell adhesion promoter crosslinked with PEG and (ii) nanoparticles. Preferably, the nanoparticles are dispersed throughout the scaffold. Over time, the scaffold degrades (e.g., via dissolution and/or mechanical degradation) and the surrounding tissue grows in its place. The nanoparticles are preferably metal oxide nanoparticles that also degrade during scaffold degradation. In such instances, the dissolution of the metal oxide nanoparticles may result in metal cations (e.g., Mg²⁺) that promote cell growth and tissue regeneration.

### Nanocomposite Hydrogel Precursors

The nanocomposite hydrogel precursor A may comprise an N-hydroxysuccinimide-terminal polyethylene glycol (PEG-NHS) in a carrier fluid.

PEG-NHS includes a polyethylene glycol polymer with multiple NHS-terminal groups (e.g., two or more NHS-terminal groups, three or more NHS-terminal groups, or 4 or more NHS-terminal groups). Examples of PEG-NHS may include, but are not limited to, 4-arm N-hydroxysuccinimide-terminal polyethylene glycol (4-arm-PEG-NHS), 6-arm N-hydroxysuccinimide-terminal polyethylene glycol (6-arm-PEG-NHS), 8-arm N-hydroxysuccinimide-terminal polyethylene glycol (8-arm-PEG-NHS), and hyperbranched PEG-NHS with multiple NHS functional groups (>12), among others. Combinations of two or more of the foregoing example PEG-NHS may be used.

The PEG-NHS may have a molecular weight ranging from about 5,000 g/mol to about 75,000 g/mol (e.g., about 5,000 g/mol to about 45,000 g/mol, about 30,000 g/mol to about 60,000 g/mol, or about 45,000 g/mol to about 75,000 g/mol). Unless otherwise specified, molecular weight is number average molecular weight (Mn) determined by light scattering according to ASTM D4001-20.

The PEG-NHS may be present in the nanocomposite hydrogel precursor A at a concentration of about 4 wt % to about 20 wt % (e.g., about 4 wt % to about 15 wt %, about 6 wt % to about 10 wt %, or about 15 wt % to about 20 wt %), based on a total weight of the nanocomposite hydrogel precursor A.

Examples of carrier fluids suitable for use in the nanocomposite hydrogel precursor A may include, but are not limited to, water, saline, phosphate buffered saline (PBS), and the like.

The nanocomposite hydrogel precursor A may have a pH of about 7 to about 9 (e.g., about 7 to about 8, about 7.2 to about 7.6, about 7.5 to about 8.5, or about 8 to about 9).

The nanocomposite hydrogel precursor A may further comprise one or more additives. Additives should not facilitate hydrolysis of the NHS-terminal groups of the PEG-NHS. Examples of additives for the nanocomposite hydrogel precursor A may include, but are not limited to, drugs, pro-drugs, nutraceuticals, antibiotics, anti-inflammatories, analgesics (e.g., acetaminophen), the like, and any combination thereof.

The nanocomposite hydrogel precursor B may comprise a serum albumin, a cell adhesion promoter, and a nanoparticle in a carrier fluid.

Examples of serum albumin may include, but are not limited to, bovine serum albumin (BSA), human serum albumin (HSA), the like, and any combination thereof.

The serum albumin may be present in the nanocomposite hydrogel precursor B at a concentration of about 4 wt % to about 20 wt % (e.g., about 4 wt % to about 15 wt %, about 6 wt % to about 10 wt %, or about 15 wt % to about 20 wt %), based on a total weight of the nanocomposite hydrogel precursor B.

Examples of cell adhesion promoters may include, but are not limited to, amino-arginyl aspartic acid peptide (amino-RGD), poly-L-lysine (PLL), poly-D-lysine (PDL), KQAGDV peptide, VAPG peptide, FGL peptide, peptides parathyroid hormone, calcitonin gene-related peptide, osteogenic growth peptide, fibronectin, elastin, collagen, laminin, the like, and any combination thereof.

The cell adhesion promoters may be present in the nanocomposite hydrogel precursor B at a concentration of about 0.01 wt % to about 20 wt % (e.g., about 0.01 wt % to about 3 wt %, about 1 wt % to about 5 wt %, about 3 wt % to about 15 wt %, or about 10 wt % to about 20 wt %), based on a total weight of the nanocomposite hydrogel precursor B.

The nanoparticles may be metal oxide nanoparticles, metal nanoparticles, or a combination of both metal oxide nanoparticles and metal nanoparticles. Examples of metal oxides in the metal oxide nanoparticles may include, but are not limited to, magnesium oxide, calcium oxide, aluminum oxide, zirconium oxide, nanohydroxyapatite, silica, and bio-ceramics, the like, and any combination thereof. Gold or other bio-inert metals may be used in the metal nanoparticles. In the examples herein, magnesium oxide (MgO) nanoparticles were used.

The nanoparticles may have an average diameter of about 1 nm to about 100 nm (e.g., about 1 nm to about 50 nm, about 5 nm to about 30 nm, or about 50 nm to about 100 nm). Unless otherwise specified, the average diameter is a weight-based average diameter.

The nanoparticles may be present in the nanocomposite hydrogel precursor B at a concentration of about 1 mg/mL to about 200 mg/mL (e.g., about 1 mg/mL to about 100 mg/mL, about 50 mg/mL to about 150 mg/mL, or about 100 mg/mL to about 200 mg/mL), based on a total volume of the nanocomposite hydrogel precursor B.

Examples of carrier fluids suitable for use in the nanocomposite hydrogel precursor B may include, but are not limited to, water, saline, phosphate buffered saline (PBS), and the like.

The nanocomposite hydrogel precursor B may further comprise one or more additives. Examples of additives for the nanocomposite hydrogel precursor A may include, but are not limited to, drugs, pro-drugs, nutraceuticals, antibiotics, anti-inflammatoires, analgesics (e.g., acetaminophen), the like, and any combination thereof.

Examples of growth factors may include, but are not limited to, bone morphogenetic protein-2 (BMP-2), fibroblast growth factor-2 (FGF-2), nerve growth factor (NGF), vascular endothelial growth factor (VEGF), the like, and any combination thereof.

The nanocomposite hydrogel precursor B may have a pH of about 7 to about 9 (e.g., about 7 to about 8, about 7.2 to about 7.6, about 7.5 to about 8.5, or about 8 to about 9).

### Nanocomposite Hydrogels, Methods of Production, and Related Applications

The nanocomposite hydrogels of the present disclosure may be formed by mixing the nanocomposite hydrogel precursor A and the nanocomposite hydrogel precursor B and allowing the PEG-NHS to crosslink the serum albumin and the cell adhesion promoter. The resultant nanocomposite hydrogel comprises (i) a scaffold comprising serum albumin and cell adhesion promoter crosslinked with PEG and (ii) a nanoparticle. See FIG. 1. The PEG is bound to the serum albumin and cell adhesion promoter via amide bonds. The scaffold may further include serum albumin and/or cell adhesion promoter that is not crosslinked.

The concentrations of PEG-NHS (crosslinked or otherwise), serum albumin (crosslinked or otherwise), cell adhesion promoter, and nanoparticles in the nanocomposite hydrogel may be any range resulting from the concentrations of individual components in the nanocomposite hydrogel precursor A and B and the ratio of nanocomposite hydrogel precursor A to B described herein. The following provide preferred ranges. However, concentrations outside the following ranges are contemplated.

The PEG-NHS (crosslinked or otherwise) may be present in the nanocomposite hydrogel at a concentration of about 0.6 wt % to about 16.7 wt % (e.g., about 0.6 wt % to about 2 wt %, about 1 wt % to about 10 wt %, about 6 wt % to about 12 wt % or about 12 wt % to about 16.7 wt %), based on a total weight of the nanocomposite hydrogel.

The serum albumin (crosslinked or otherwise) may be present in the nanocomposite hydrogel at a concentration of about 0.6 wt % to about 16.7 wt % (e.g., about 0.6 wt % to about 2 wt %, about 1 wt % to about 10 wt %, about 6 wt % to about 12 wt % or about 12 wt % to about 16.7 wt %), based on a total weight of the nanocomposite hydrogel. Preferably, at least 80 wt % (e.g., about 80 wt % to 100 wt %, about 85 wt % to 100 wt %, about 90 wt % to 100 wt %, about 95 wt % to 100 wt %, or about 98 wt % to 100 wt %) of the serum albumin is crosslinked.

The cell adhesion promoter may be present in the nanocomposite hydrogel at a concentration of about 0.00017 wt % to about 16.7 wt %, (e.g., about 0.00017 wt % to about 0.05 wt %, about 0.01 wt % to about 0.5 wt %, about 0.5 wt % to about 4 wt %, about 4 wt % to about 10 wt % or about 10 wt % to about 16.7 wt %), based on a total weight of the nanocomposite hydrogel. Preferably, at least 80 wt % (e.g., about 80 wt % to 100 wt %, about 85 wt % to 100 wt %, about 90 wt % to 100 wt %, about 95 wt % to 100 wt %, or about 98 wt % to 100 wt %) of the cell adhesion promoter is crosslinked.

The nanoparticles may be present in the nanocomposite hydrogel at a concentration of 0.17 mg/mL to about 166.7 mg/mL (e.g., about 0.17 mg/mL to about 10 mg/mL, about 10 mg/mL to about 75 mg/mL, about 60 mg/mL to about 100 mg/mL or about 100 mg/mL to about 166.7 mg/mL), based on a total volume of the nanocomposite hydrogel.

When mixing the precursors, the volume ratio of the nanocomposite hydrogel precursor A to the nanocomposite hydrogel precursor B may range from about 5:1 to about 1:5 (e.g., about 4:1 to about 1:4, about 3:1 to about 1:3, or about 2:1 to about 1:2).

After mixing nanocomposite hydrogel precursor A and the nanocomposite hydrogel precursor B, the resultant mixture may have a pH of about 7 to about 9 (e.g., about 7 to about 8, about 7.2 to about 7.6, about 7.5 to about 8.5, or about 8 to about 9).

The method of mixing may depend on, among other things, the gelation time of the nanocomposite hydrogel. For example, long gelation times may allow for mixing the precursors in one vessel and then transferring the mixture before gelation is complete to desired location (e.g., a space in a biological environment, a mold, or the like) for the nanocomposite hydrogel.

In another example, short gelation times may facilitate injection directly into the desired location for the nanocomposite hydrogel where the precursors are physically separated (e.g., in separate containers) and injection into the location facilitates mixing of the precursors. Accordingly, an injectable composition of the present disclosure may include a nanocomposite hydrogel precursor A and a nanocomposite hydrogel precursor B, where the precursors are physically separated from each other.

The nanocomposite hydrogel may be characterized by its gelation time. Determination of the gelation time is described in the examples section. A nanocomposite hydrogel may have a gelation time of about 3 seconds to 30 minutes (e.g., 3 seconds to 1 minute, 10 seconds to 5 minutes, 30 seconds to 5 minutes, 1 minute to 20 minutes, or 5 minutes to 30 minutes).

The gelation kinetics of the nanocomposite hydrogel is influenced by temperature. However, amidation reactions typically occur at elevated temperatures to facilitate the reaction kinetics, commonly ranging from 60° C. to 100° C. However, it may be preferable for the gelation process to occur at room temperature or at the physiological body temperature of 37° C. considering the convenient fabrication and clinical application.

The nanocomposite hydrogel exhibits a white texture and a rough surface, with surface properties that vary depending on the addition of nanoparticles. At the microscale, the nanocomposite hydrogel displays a porous structure with tunable pore sizes. In contrast, the nanoparticle-free control hydrogel (without nanoparticles, see examples) has a transparent texture and a smooth surface.

Nanocomposite hydrogels may be formed and/or used in in vivo environments, in vitro environments, or non-biological environments. For example, a nanocomposite hydrogel may be formed directly in a desired in vivo environment (e.g., a space within a tissue or bone) for treating and/or repairing the tissue and/or bone in which the nanocomposite hydrogel is formed. In another example, a nanocomposite hydrogel may be formed into a desired shape in vitro and seeded with stem cells before in vivo implantation. In yet another example, the nanocomposite hydrogel may be made and used for non-biological applications.

For in vivo applications, the nanocomposite hydrogel may be formed and/or placed, after in vitro formation, in damaged or diseased tissues such as cardiac, bone, liver, corneal and skin tissues. The nanocomposite hydrogel can have a drug, pro-drug, the like, or a combination thereof that promotes tissue repair and growth. Further, the nanocomposite hydrogel may encapsulate the stem cells and exosomes therein. For example, an in vitro nanocomposite hydrogel may be impregnated with cells (e.g., stem cells) before implantation. In another example, after gelation of a nanocomposite hydrogel formed in vivo, the nanocomposite hydrogel may be impregnated with cells.

The present disclosure also includes kits for producing nanocomposite hydrogel. The kit may include (i) nanocomposite hydrogel precursor A or a portion thereof, (ii) nanocomposite hydrogel precursor B or a portion thereof, and (iii) a set of instructions for producing the nanocomposite hydrogel. For example, the PEG-NHS may be prone to hydrolysis and lose efficacy over time. Accordingly, the PEG-NHS may be in the kit as a solid that is mixed with a carrier fluid to form the nanocomposite hydrogel precursor A within an allotted time before mixing the two precursors. Similarly, the nanocomposite hydrogel precursor B may be provided in the kit as the solid material that is mixed with a carrier fluid within an allotted time before mixing the two precursors. Said carrier fluids for either or both precursors may be present in the kit or provided by the user.

Additional elements of the kit may include, but are not limited to, a container for preparing the nanocomposite hydrogel precursor A, a container for preparing the nanocomposite hydrogel precursor B, a container for mixing the precursors therein, a mixing apparatus (e.g., a double syringe) that physically separates the precursors and can effect mixing thereof, pH paper, sterilization equipment (e.g., a filter or UV light), the like, and any combination thereof.

Unless otherwise indicated, all numbers expressing quantities of ingredients, properties (e.g., molecular weight), reaction conditions, and the like used in the present disclosure and associated claims are to be understood as being modified in all instances by the term “about.” At the very least, and not as an attempt to limit the application of the doctrine of equivalents to the scope of the claim, the term “about” relative to each numerical parameter should at least be construed considering the number of reported significant digits and by applying ordinary rounding techniques.

A concentration range listed or described as being useful, suitable, or the like, is intended that any and every concentration within the range, including the end points, is to be considered as having been stated. For example, a range “from 1 to 10” or “of 1 to 10” is to be read as indicating each and every possible number along the continuum between about 1 and about 10. Thus, even if specific data points within the range, or even no data points within the range, are explicitly identified or refer to only a few specific data points, it is to be understood that inventors appreciate and understand that any and all data points within the range are to be considered to have been specified, and that inventors possessed knowledge of the entire range and all points within the range.

The term “and/or” refers to both the inclusive “and” case and the exclusive “or” case, and is used herein for brevity. For example, a mixture comprising acetic acid and/or methyl acetate may comprise acetic acid alone, methyl acetate alone, or both acetic acid and methyl acetate.

A listing following “one or more of” or “at least one of” using “and” to connect the listing is intended in the alternative or conjunctive rather than the disjunctive. For example, “at least one of: A, B, and C” and “one or more of: A, B, and C” are each considered to disclose embodiments of A alone, B alone, C alone, A and B in combination, A and C in combination, B and C in combination, and all three of A, B, and C in combination.

Room temperature is 25° C., and atmospheric pressure is 101.325 kPa unless otherwise noted.

While compositions, systems, and methods are described herein in terms of “comprising” various components or steps, the compositions, systems, and methods can also “consist essentially of” or “consist of” the various components and steps.

### Example Embodiments

Embodiment 1. An injectable composition comprising: a nanocomposite hydrogel precursor A comprising that comprises a polyethylene glycol with two or more N-hydroxysuccinimide-terminal groups (PEG-NHS); and a nanocomposite hydrogel precursor B comprising a serum albumin, a nanoparticle, and a cell adhesion promoter; wherein the nanocomposite hydrogel precursor A and the nanocomposite hydrogel precursor B are physically separated; and wherein the nanocomposite hydrogel precursor A and the nanocomposite hydrogel precursor B are capable of forming a nanocomposite hydrogel when mixed.

Embodiment 2. The injectable composition of Embodiment 1, wherein the PEG-NHS is a 4-arm-PEG-NHS, a 6-arm-PEG-NHS, an 8-arm-PEG-NHS, hyperbranched PEG-NHS, or any combination thereof.

Embodiment 3. The injectable composition of any one of Embodiments 1-2, wherein the PEG-NHS has a molecular weight of 5,000 g/mol to 75,000 g/mol.

Embodiment 4. The injectable composition of any one of Embodiments 1-3, wherein the PEG-NHS is present at 4 wt % to 20 wt %, based on a total weight of the nanocomposite hydrogel precursor A.

Embodiment 5. The injectable composition of any one of Embodiments 1-4, wherein the serum albumin is present at 4 wt % to 20 wt %, based on a total weight of the nanocomposite hydrogel precursor B.

Embodiment 6. The injectable composition of any one of Embodiments 1-5, wherein the nanoparticle comprises magnesium oxide, calcium oxide, aluminum oxide, zirconium oxide, nanohydroxyapatite, silica, a bio-ceramic, gold, or any combination thereof.

Embodiment 7. The injectable composition of any one of Embodiments 1-6, wherein the nanoparticle has an average diameter of 1 nm to 100 nm.

Embodiment 8. The injectable composition of any one of Embodiments 1-7, wherein the nanoparticle is present at 1 mg/mL to 200 mg/mL, based on a total volume of the nanocomposite hydrogel precursor B.

Embodiment 9. The injectable composition of any one of Embodiments 1-8, wherein the cell adhesion promoter comprises a peptide or protein.

Embodiment 10. The injectable composition of Embodiment 9, wherein the peptide comprises amino-arginyl aspartic acid peptide (amino-RGD), a KQAGDV peptide, a VAPG peptide, a FGL peptide, calcitonin gene-related peptide, osteogenic growth peptide, fibronectin, elastin, collagen, laminin, or any combination thereof.

Embodiment 11. The injectable composition of any one of Embodiments 1-10, wherein the cell adhesion promoter is present at 0.01 wt % to 20 wt %, based on a total weight of the nanocomposite hydrogel precursor B.

Embodiment 12. The injectable composition of any one of Embodiments 1-11, wherein, when mixed, the nanocomposite hydrogel precursor A and the nanocomposite hydrogel precursor B are capable of forming the nanocomposite hydrogel having a gelation time of 30 minutes or less.

Embodiment 13. The injectable composition of any one of Embodiments 1-12, wherein the nanocomposite hydrogel precursor B further comprises a drug, a pro-drug, a nutraceutical, a growth factor, or any combination thereof.

Embodiment 14. A method comprising: injecting a nanocomposite hydrogel precursor A and a nanocomposite hydrogel precursor B into a space in a biological environment, thereby causing mixing thereof and formation of a nanocomposite hydrogel in the space, wherein the nanocomposite hydrogel precursor A comprises a polyethylene glycol with two or more N-hydroxysuccinimide-terminal groups (PEG-NHS), and wherein the nanocomposite hydrogel precursor B comprises a serum albumin, a nanoparticle, and a cell adhesion promoter.

Embodiment 15. The method of Embodiment 14, wherein the nanocomposite hydrogel has a gelation time of 30 minutes or less.

Embodiment 16. The method of any one of Embodiments 14-15, wherein the space is a defect in a tissue.

Embodiment 17. The method of Embodiment 16, wherein the tissue is bone.

Embodiment 18. The method of any one of Embodiments 14-17, wherein a volume ratio of the nanocomposite hydrogel precursor A and the nanocomposite hydrogel precursor B is 5:1 to 1:5.

Embodiment 19. A method comprising: mixing a nanocomposite hydrogel precursor A and a nanocomposite hydrogel precursor B to form a nanocomposite hydrogel, wherein the nanocomposite hydrogel precursor A comprises N-a polyethylene glycol with two or more N-hydroxysuccinimide-terminal groups (PEG-NHS), and wherein the nanocomposite hydrogel precursor B comprises a serum albumin, a nanoparticle, and a cell adhesion promoter.

Embodiment 20. A hydrogel composition comprising: a scaffold comprising serum albumin and a cell adhesion promoter crosslinked with polyethylene glycol; and a nanoparticle dispersed in the scaffold.

Hereinafter, the present invention will be better understood in view of the following nonlimiting examples.

### Examples

Preparation of Nanocomposite Hydrogels. A nanocomposite hydrogel precursor A was prepared having 8 wt % 4-arm-PEG-NHS in PBS. A nanocomposite hydrogel precursor B was prepared by first adding 2 mg/mL of amino-RGD to a solution of 10 wt % BSA in PBS buffer. The resulting solution was then filtered through a microporous membrane (0.22 μm) for sterilization. MgO nanoparticles were then added to the sterile solution of amino-RGD and BSA in PBS at various concentrations from 1 mg/mL to 30 mg/mL to produce a series of nanocomposite hydrogel precursors B (NHP-B). In a 1:1 volumetric ratio, nanocomposite hydrogel precursor A was mixed with each of the nanocomposite hydrogel precursors B. The mixtures were allowed to rest without agitation for several minutes to form nanocomposite hydrogels. The nanocomposite hydrogels were sealed, protected from light, and stored at 4° C. in humid conditions.

Preparation of Control Hydrogels. Control 1 was a conventional poly(ethylene glycol) diacrylate hydrogel (PEGDA). Control 1 was prepared with 8 wt % PEGDA plus 0.1 wt % LAP photoinitiator (a water soluble photoinitiator for use in the polymerization of hydrogels or bioinks, available from Advanced BioMatrix) that was crosslinked by exposure to blue light for about 30 seconds.

Control 2 was prepared with the same method the nanocomposite hydrogels above but with no MgO nanoparticles. Hydrogel precursor A was 8 wt % 4-arm-PEG-NHS in PBS. Hydrogel precursor B was a sterilized solution of 2 mg/mL of amino-RGD and 10 wt % BSA in PBS buffer. The pH of precursor B was 7.4. After mixing hydrogel precursors A and B, the mixture was allowed to crosslink for about 30 minutes at about 37° C.

Control 3 was prepared the same as Control 2 but with a pH of 8.6 of precursor B.

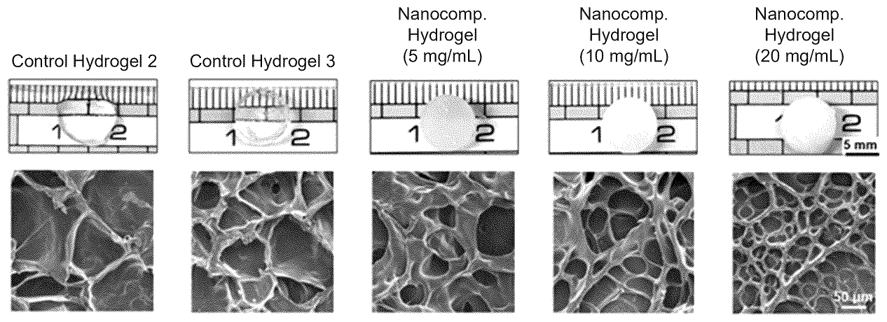

Gel Structure. A series of hydrogels (control hydrogels and nanocomposite hydrogels) were by cylinder molds each prepared according to the method above with a total volume after mixing the two precursors of about 100 μL. The resultant hydrogels were lyophilizate for 8 hours then imaged with a scanning electron microscope. FIG. 2 illustrates photographs and scanning electron micrographs corresponding to control hydrogel 2, control hydrogel 3, and nanocomposite hydrogels (5 mg/mL), nanocomposite hydrogels (10 mg/mL), and nanocomposite hydrogels (20 mg/mL), respectively.

As illustrated in the upper portions of for each sample in FIG. 2, the nanocomposite hydrogels have good structural stability. The control hydrogels are colorless and transparent. Due to the addition of nanoparticles, the nanocomposite hydrogel is white.

The scanning electrode micrographs of FIG. 2 illustrate a microscopic connected and penetrating network structure, which may help cells adhere and infiltrate the hydrogel. Without being limited by theory, it is believed that as the concentration of MgO nanoparticles increases, the pH value of the precursor solution increases, which will form more stable amide bonds and result in a denser hydrogel network.

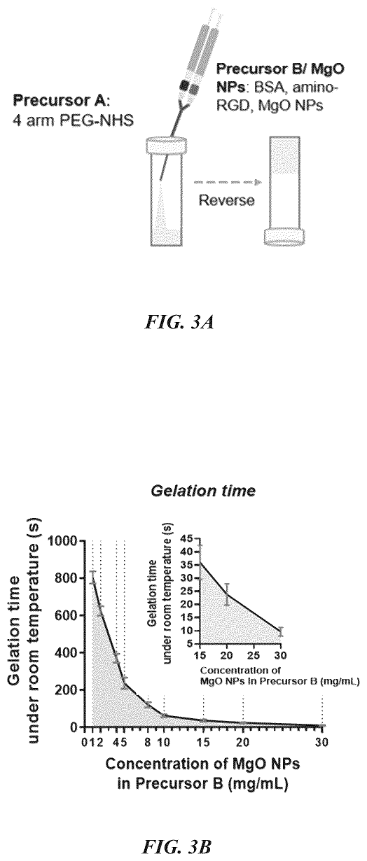

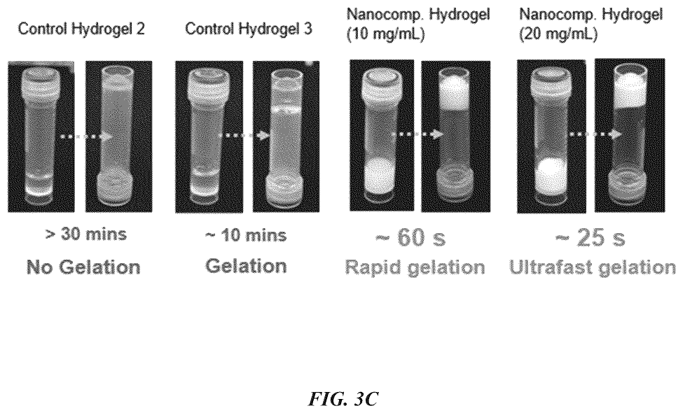

Gelation Time. A series of hydrogels (control hydrogels and nanocomposite hydrogels) were each prepared by injecting the corresponding precursors (total volume 100 μL) into a vial (inner diameter of about 1 cm) using a double barrel syringe. The gelation time was recorded as the time from mixing to when the vial could be invented with no visible liquid flow. FIG. 3A illustrates the method for ascertaining the gelation time. The gelation time was evaluated under room temperature conditions.

FIG. 3B illustrates a plot of the gelation time as a function of the MgO nanoparticle concentration where the inlay plot is the 15-30 mg/mL data enlarged. The gelation time ranges from about 800 seconds at 1 mg/mL to about 10 seconds at 30 mg/mL.

FIG. 3C illustrates photographs during the gelation time method for control hydrogels and nanocomposite hydrogels according to at least some embodiments of the present disclosure. Control 2 formed a gel-like structure by was not completely gelled after 30 minutes. That is, upon inversion of the vial, fluid still flowed in Control 2. The gelation time for Control 3 was about 600 seconds. This example illustrates that the gelation time of the nanocomposite hydrogels of the present disclosure can be controlled by controlling the nanoparticle concentration, which may be advantageous for clinical microsurgery (injection) implantation.

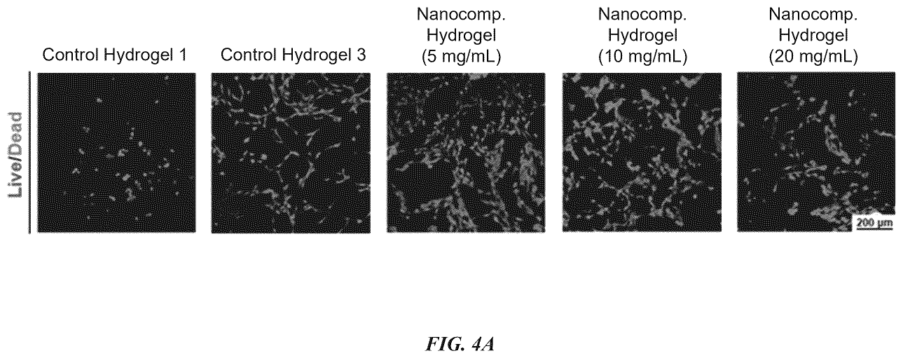

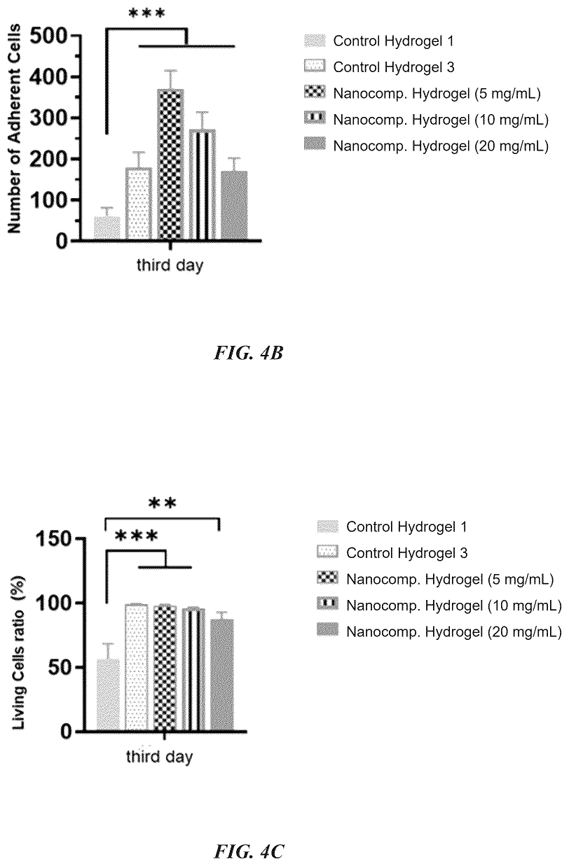

In Vitro Biocompatibility. A series of hydrogels (control hydrogels and nanocomposite hydrogels) were prepared as discussed above. After gelation, the hydrogels were soaked in PBS and sterilized with ultraviolet light for one hour. The hydrogels were then transferred to wells within a 24-well plate to which about 5×10⁴ rat-derived mandible marrow mesenchymal stem cells (MMSCs) were added with 500 μL of MEM medium. The hydrogels and cells were incubated for 3 days at 37° C., 100% humidity, and 5% CO₂. Then, a Live/Dead cell imaging kit (available from ThermoFisher) was used to stain the cells. A confocal microscope was used to observe the adherent cells and count the live and dead cells. FIG. 4A illustrates a fluorescent image after treating with the Live/Dead cell imaging kit for each sample.

FIG. 4B is a bar graph of the number of adherent cells after the 3-day incubation period (*P<0.05, **P<0.01, ***P<0.001). FIG. 4C is a bar graph of the percentage of total cells that are living after the 3-day incubation period (*P<0.05, **P<0.01, ***P<0.001). Compared with conventional poly(ethylene glycol) diacrylate hydrogel (control hydrogel 1), the control hydrogel 3 and the nanocomposite hydrogels have amino cell adhesion sequences and biological proteins added thereto to further improve biocompatibility and enhance interactions between cells and hydrogels. As shown in FIGS. 4B and 4C, more MMSCs adhered and spread on the hydrogel surface where said additional components were included. Further, the presence of MgO nanoparticles also significantly increased cell adhesion and proliferation on nanocomposite hydrogels. Both the control hydrogel 3 and the nanocomposite hydrogels exhibited a higher proportion of living cells compared to the control hydrogel 1.

Establishment of Drug-Related Jaw Osteonecrosis and Necrosis Debridement in Rat Model. Experimental rats received weekly intraperitoneal injection of zoledronic acid (ZOL) at a dose of 300 μg/kg for 12 weeks. One week after completion of ZOL administration, surgical intervention was performed. After anesthesia, an incision was made parallel to the outer edge of the lower border of the left mandible, and the subcutaneous tissue and masseter muscle were bluntly dissected to expose the mandible. A penetrating circular hole with a diameter of 3 mm was created under the first and second molars using a surgical drill. After flushing the surgical area with physiological saline, the sterilized hydrogels (control hydrogels and nanocomposite hydrogels) were implanted making sure the hydrogel fully fills the fractured tissue. Absorbable sutures were used to suture the muscles, and silk sutures were used to suture the skin.

Verification of the Efficacy of Nanocomposite Hydrogel in Bone Defect Regeneration. 4, 8, and 12 weeks after surgery, the animals were killed by intraperitoneal injection of pentobarbital, and samples from the mandible defect area were collected and treated with 4% poly Formaldehyde fixation. X-rays, micro-computed tomography (micro-CT) analysis, and angiographic analysis of new bone in the bone defect are performed.

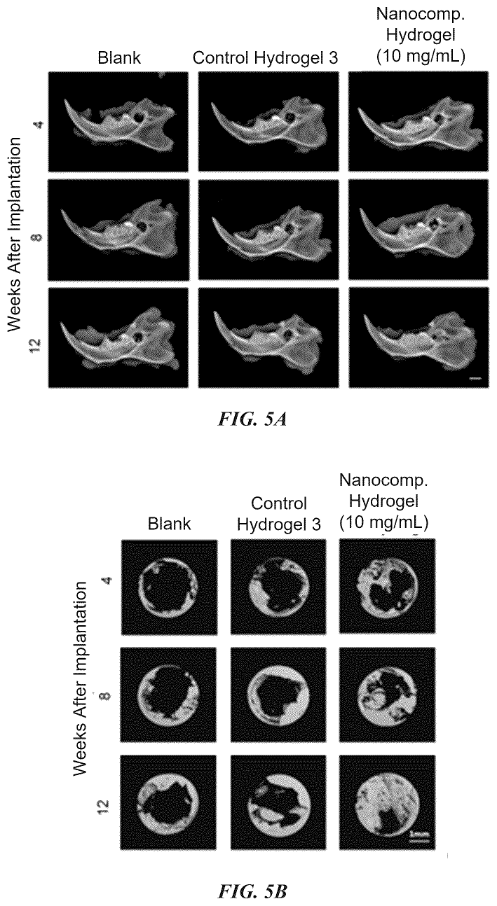

X-ray images (FIG. 5A whole mandible) were taken from the 4, 8 and 12 weeks after implantation. The bone around the mandible defect with the nanoparticle hydrogel group (10 mg/mL MgO nanoparticles) thickened, and new bone gradually appeared inside the defect. By 12 weeks, new bone was grown into the nanocomposite hydrogel and filled a large area of the defect area. In contrast, a blank control group (no hydrogel implantation) and the control hydrogel group (control hydrogel 3) showed insufficient new bone formation. Micro-CT three-dimensional reconstruction were also prepared, which consistently showed the foregoing trends (FIG. 5B drilled hole).

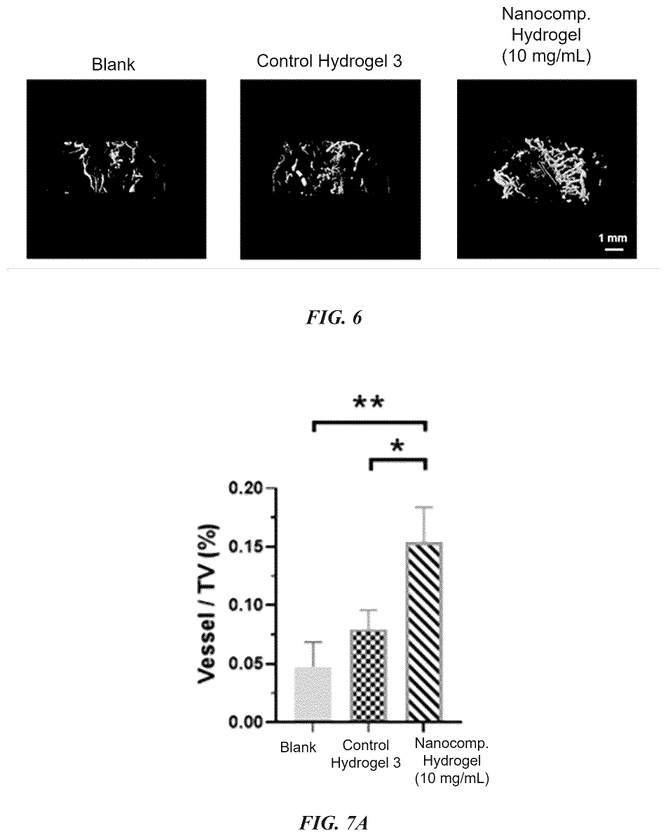

FIG. 6 illustrates three-dimensional reconstructions of micro-CT based angiography for the three groups (blank, control hydrogel 3, and nanocomposite hydrogel with 10 mg/mL MgO nanoparticles). Based on these reconstructions, the percentage of blood vessel volume and the number of blood vessels per unit area in the nanocomposite hydrogel group at 8 weeks are significantly higher than those in the blank control group and the control hydrogel 3 group.

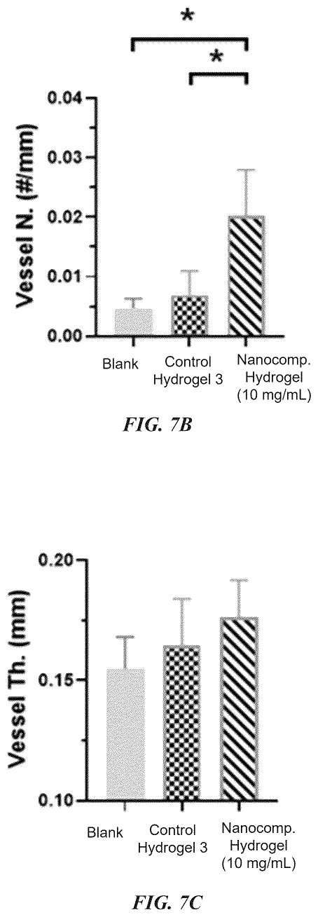

FIG. 7A is a bar graph of the percentage of blood vessels based on the total volume of the defect area. FIG. 7B is a bar graph of the number of blood vessels in the defect area. FIG. 7C is a bar graph of the thickness of the blood vessels. There was no significant difference in the number of blood vessels between the control hydrogel group and the blank control group, which means that the nanocomposite hydrogel of this product can repair bone defects by enhancing blood vessels.

Illustrative embodiments of the invention are described herein. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

What is claimed is:

1. An injectable composition comprising:
a nanocomposite hydrogel precursor A comprising that comprises a polyethylene glycol with two or more N-hydroxysuccinimide-terminal groups (PEG-NHS); and
a nanocomposite hydrogel precursor B comprising a serum albumin, a nanoparticle, and a cell adhesion promoter;
wherein the nanocomposite hydrogel precursor A and the nanocomposite hydrogel precursor B are physically separated; and
wherein the nanocomposite hydrogel precursor A and the nanocomposite hydrogel precursor B are capable of forming a nanocomposite hydrogel when mixed.

2. The injectable composition of claim 1, wherein the PEG-NHS is a 4-arm-PEG-NHS, a 6-arm-PEG-NHS, an 8-arm-PEG-NHS, hyperbranched PEG-NHS, or any combination thereof.

3. The injectable composition of claim 1, wherein the PEG-NHS has a molecular weight of 5,000 g/mol to 75,000 g/mol.

4. The injectable composition of claim 1, wherein the PEG-NHS is present at 4 wt % to 20 wt %, based on a total weight of the nanocomposite hydrogel precursor A.

5. The injectable composition of claim 1, wherein the serum albumin is present at 4 wt % to 20 wt %, based on a total weight of the nanocomposite hydrogel precursor B.

6. The injectable composition of claim 1, wherein the nanoparticle comprises magnesium oxide, calcium oxide, aluminum oxide, zirconium oxide, nanohydroxyapatite, silica, a bio-ceramic, gold, or any combination thereof.

7. The injectable composition of claim 1, wherein the nanoparticle has an average diameter of 1 nm to 100 nm.

8. The injectable composition of claim 1, wherein the nanoparticle is present at 1 mg/mL to 200 mg/mL, based on a total volume of the nanocomposite hydrogel precursor B.

9. The injectable composition of claim 1, wherein the cell adhesion promoter comprises a peptide or protein.

10. The injectable composition of claim 9, wherein the peptide comprises amino-arginyl aspartic acid peptide (amino-RGD), a KQAGDV peptide, a VAPG peptide, a FGL peptide, calcitonin gene-related peptide, osteogenic growth peptide, fibronectin, elastin, collagen, laminin, or any combination thereof.

11. The injectable composition of claim 1, wherein the cell adhesion promoter is present at 0.01 wt % to 20 wt %, based on a total weight of the nanocomposite hydrogel precursor B.

12. The injectable composition of claim 1, wherein, when mixed, the nanocomposite hydrogel precursor A and the nanocomposite hydrogel precursor B are capable of forming the nanocomposite hydrogel having a gelation time of 30 minutes or less.

13. The injectable composition of claim 1, wherein the nanocomposite hydrogel precursor B further comprises a drug, a pro-drug, a nutraceutical, a growth factor, or any combination thereof.

14. A method comprising:
injecting a nanocomposite hydrogel precursor A and a nanocomposite hydrogel precursor B into a space in a biological environment, thereby causing mixing thereof and formation of a nanocomposite hydrogel in the space,
wherein the nanocomposite hydrogel precursor A comprises a polyethylene glycol with two or more N-hydroxysuccinimide-terminal groups (PEG-NHS), and
wherein the nanocomposite hydrogel precursor B comprises a serum albumin, a nanoparticle, and a cell adhesion promoter.

15. The method of claim 14, wherein the nanocomposite hydrogel has a gelation time of 30 minutes or less.

16. The method of claim 14, wherein the space is a defect in a tissue.

17. The method of claim 16, wherein the tissue is bone.

18. The method of claim 14, wherein a volume ratio of the nanocomposite hydrogel precursor A and the nanocomposite hydrogel precursor B is 5:1 to 1:5.

19. A method comprising:
mixing a nanocomposite hydrogel precursor A and a nanocomposite hydrogel precursor B to form a nanocomposite hydrogel,
wherein the nanocomposite hydrogel precursor A comprises N-a polyethylene glycol with two or more N-hydroxysuccinimide-terminal groups (PEG-NHS), and
wherein the nanocomposite hydrogel precursor B comprises a serum albumin, a nanoparticle, and a cell adhesion promoter.

20. A hydrogel composition comprising:
a scaffold comprising serum albumin and a cell adhesion promoter crosslinked with polyethylene glycol; and
a nanoparticle dispersed in the scaffold.
